SEQUENCE LISTING (2 sequences)

SEQ ID NO: 1 - protein, 92 aa, Homo sapiens
MASLGHILVFCVGLLTMAKAESPKEHDPFTYDYQSLQIGGLVIAGILFILGILIVLSRRCRCKFNQQQRTGEPDEEEGTFRSSIRRLSTRRR

SEQ ID NO: 2 - DNA, 279 bp, Homo Sapiens
atggcacctctccaccacatcttggttttctgtgtgggtctcctcaccatggccaaggcagaaagtccaaaggaacacgacccgttcacttacgactaccagtccctgcagatcggaggcctcgtcatcgccgggatcctcttcatcctgggcatcctcatcgtgctgagcagaagatgccggtgcaagttcaaccagcagcagaggactggggaacccgatgaagaggagggaactttccgcagctccatccgccgtctgtccacccgcaggcggtag

(19) United States
(12) Patent Application Publication
(10) Pub. No.: US 2018/0185444 A1
(43) Pub. Date: Jul. 5, 2018

(54) METHODS FOR TREATMENT AND PREVENTION OF VASCULAR DISEASE

(71) Applicant: Gemma Alexandra Figtree, Wollstonecraft (AU)

(72) Inventor: Gemma Alexandra Figtree, Wollstonecraft (AU)

(21) Appl. No.: 15/508,410

(22) PCT Filed: Sep. 7, 2015
(86) PCT No.: PCT/AU2015/000548

§ 371 Date: Mar. 2, 2017

(30) Foreign Application Priority Data
Sep. 5, 2014 (AU) 2014903547

(51) Int. Cl. A61K 38/17; A61L 31/16; A61L 27/50; A61L 27/54; A61K 48/00

(52) U.S. Cl. CPC A61K 38/177; A61L 31/16; A61L 27/507; A61L 2300/606; A61K 48/00; A61L 2300/252; A61L 27/54

(57) ABSTRACT

The present invention relates to methods of treatment and or prevention of vascular disease. The present invention also relates to a medical device for implantation in a patient undergoing vasculature therapy, the device comprising a therapeutic amount of FXYD1 or a derivative thereof capable of interaction with endothelial nitric oxide synthase. The present invention also relates to the use of FXYD1 and derivatives and variants thereof capable of interaction with endothelial nitric oxide synthase for the treatment or prevention of vascular disease.

### RELATED APPLICATION

This application claims priority from Australian Provisional Patent Application No. 2014903547 entitled “Method for treatment and prevention of vascular disease”, filed 5 Sep. 2014, the contents of which are incorporated by reference.

### FIELD

The present invention relates to methods of treatment and or prevention of vascular disease. The present invention also relates to a medical device for implantation in a patient undergoing vasculature therapy, the device comprising a therapeutic amount of FXYD1 or a derivative thereof capable of interaction with endothelial nitric oxide synthase. The present invention also relates to the use of FXYD1 and derivatives and variants thereof capable of interaction with endothelial nitric oxide synthase for the treatment or prevention of vascular disease.

### BACKGROUND

Abnormal vascular function is a key feature of hypertension, atherosclerosis, diabetes and ageing, and, as such, is a major contributor to morbidity and mortality. A common factor driving vascular dysfunction in these disease states is the excess generation of reactive oxygen species (ROS). Superoxide anions (O₂^{⋅-}) and related ROS not only quench nitric oxide (NO), but they also directly impair the function of cellular proteins via oxidative post-translational modification, driving inflammation, cell proliferation, fibrosis, atherosclerosis and impairments in membrane transport. A major contributor to this elevated ROS in vascular disease is the activation of the renin-angiotensin system, resulting in Angiotensin II (Ang II)-activation of NADPH oxidase, as well as uncoupling of endothelial nitric oxide synthase (eNOS).

There remains a need for new or improved methods and agents for preventing or reducing abnormal vascular function, and for treating or preventing medical conditions associated with abnormal vascular function.

### SUMMARY OF INVENTION

The inventors have surprisingly identified that FXYD1 is an endogenous protector of endothelial nitric oxide synthase (eNOS) against redox induced uncoupling, resulting in rapid improvements in nitric oxide (NO) bioavailability, reduction in redox stress, and improvements in chronic NO-dependent phenotypes of medial hypertrophy and perivascular fibrosis. Supplementation of FXYD1, for example via recombinant protein administration (of native FXYD1 or engineered derivatives) or gene-transfer is a novel therapy that will protect the artery from redox-induced dysfunction in a wide range of vascular disease states characterized by oxidative stress (summarized in FIG. 1).

Accordingly, in one aspect, the invention provides a method for the treatment or prevention of redox-induced dysfunction of the vasculature, the method comprising administering to a patient having or at risk of redox-induced dysfunction of the vasculature a therapeutically effective amount of FXYD1 or a derivative or variant thereof capable of interaction with endothelial nitric oxide synthase.

In an embodiment administering FXYD1 or a derivative thereof capable of interaction with endothelial nitric oxide synthase comprises delivery of a nucleic acid sequence encoding FXYD1 or a derivative thereof capable of interaction with endothelial nitric oxide synthase to selected vasculature of said patient. In an embodiment administering FXYD1 or a derivative thereof capable of interaction with endothelial nitric oxide synthase comprises delivery of a viral vector to selected vasculature of said patient, the viral vector encoding FXYD1 or a derivative thereof capable of interaction with endothelial nitric oxide synthase.

In an embodiment the method comprises administration of said FXYD1 or a derivative thereof capable of interaction with endothelial nitric oxide synthase to a vessel during surgical or interventional procedure on said patient. In an embodiment administration to said patient comprises incubating, in a composition comprising FXYD1 or a derivative or variant thereof capable of interaction with endothelial nitric oxide synthase, a coronary artery bypass graft prior to anastomosis during surgery. In an embodiment administration to said patient comprises implantation in said patient of a coated vessel or coated stent, said coating comprising FXYD1 or a derivative thereof capable of interaction with endothelial nitric oxide synthase (eNOS).

In one aspect the invention provides a medical device for implantation in a patient undergoing vasculature therapy, the device comprising a therapeutic amount of FXYD1 or a derivative thereof capable of interaction with endothelial nitric oxide synthase. In one aspect the invention provides a medical device for implantation in a patient undergoing vasculature therapy, the device comprising a nucleic acid sequence encoding FXYD1 or a derivative thereof capable of interaction with endothelial nitric oxide synthase.

In an embodiment the device is a stent. In an embodiment the device is a coated stent, the coating comprising FXYD1 or a derivative thereof capable of interaction with endothelial nitric oxide synthase. In an embodiment the device is a coated vascular stent, such as a coronary artery stent. The medical device may comprise a naturally-occurring component(s), such as vascular tissue or blood vessel, synthetic component(s), such as a manufactured stent, or a combination thereof. In an embodiment the device is a vascular graft, which may comprise naturally occurring material, synthetic material, or a combination thereof. In an embodiment the device may comprise a drug-eluting stent or graft.

In an embodiment the patient has a condition associated with endothelial dysfunction. In an embodiment the patient has a condition selected from the group consisting of myocardial infarction, diabetes, such as diabetic peripheral vascular disease, coronary artery disease, dysfunction in the coronary, peripheral or brain circulation, chronic renal failure, such as with arterial-venous fistulas, acute cerebrovascular accident (stroke), ischaemia-reperfusion injury, chronic vascular disease, pulmonary hypertension, neuromuscular disease.

In a further aspect the invention provides FXYD1 or a derivative thereof capable of interaction with endothelial nitric oxide synthase for the treatment or prevention of redox-mediated dysfunction of the vasculature.

In a further aspect the invention provides FXYD1 or a derivative thereof capable of interaction with endothelial nitric oxide synthase for manufacture of a medicament for the treatment or prevention of redox-mediated dysfunction of the vasculature.

The summary of the invention described above is not limiting and other features and advantages of the invention will be apparent from the following detailed description of the preferred embodiments, as well as from the claims.

### BRIEF DESCRIPTION OF DRAWINGS

A preferred embodiment of the present invention will now be described, by way of an example only, with reference to the accompanying drawings wherein:

FIG. 1. Schematic representation of the protective action of FXYD1 against eNOS-uncoupling in endothelial cells. Ang II-induces NADPH oxidase-dependent eNOS uncoupling via glutathionylation in endothelial cells (Galougahi et al., 2014). NADPH oxidase (NOX)-derived O₂^{⋅-}, under Ang II type 1 receptor (AT1R)-coupled activation, initiates eNOS uncoupling by glutathionylation of critical Cys residues (C689 and C908) in the reductase domain (shown as G), leading to a decrease in NO and amplification of O₂^{⋅-} production (upper panel). This is supports the paradigm of NOX— derived O₂^{⋅-} acting as the “kindling”, and uncoupled eNOS the “bonfire”, with eNOS glutathionylation the critical molecular switch. FXYD1 protects eNOS from glutathionylation (lower panel), and resulting uncoupling, dramatically improving NO bioavailability, and reducing cellular oxidative stress in the endothelium.

FIG. 2. FXYD1 is expressed in human umbilical vein endothelial cells (HUVECs-seen in total lysate-TL), and co-immunoprecipitates with eNOS. IP=immunoprecipitant. IP of negative IgG is shown as a control.

FIG. 3. eNOS glutathionylation is increased when endogenous expression of FXYD1 is silenced.

FIG. 4. FXYD1 expression is critical for eNOS activation. When FXYD1 is silenced or “knocked down” (KD), the NO production by HUVECs is dramatically reduced as assessed by the NO-sensitive fluorescent agent DAF-2DA.

FIG. 5. Histogram demonstrating the effect of silencing FXYD1 expression with siRNA on the superoxide-sensitive dihydroethidium (DHE) fluorescence in cells exposed to Ang 11 (500 nM).

FIG. 6. FXYD1 knockout (mouse model) results in augmentation of Angiotensin II induced eNOS-glutathionylation in vivo (1 week of Ang II infusion at 1 mg/kg/day). eNOS glutathionylation is determined using GSH epitope IP, and eNOS immunoblot (IB). This is observed in both the heart (A) and the aorta (B). Size markers shown on the left side of FIG. 6A represent 10, 15, 20, 25, 37, 50, 75, 100, 150 and 250 kDa.

FIG. 7. Knockout of FXYD1 (FXYDKO) results in increased medial thickness, which is exacerbated with administration of Angiotensin II.

FIG. 8. FXYD1 knockout results in increased perivascular fibrosis, and substantial augmentation of Angiotensin II-induced perivascular fibrosis.

FIG. 9. FXYD1 knockout (mouse model) results in increased basal interstitial fibrosis and augments Angiotensin H induced interstitial fibrosis in the heart. Fibrosis is visualized using Milligans trichrome staining on fixed myocardial tissue. ** reflects significance where p<0.01.

FIG. 10. FXYD1 knockout (mouse model) results in elevated pulmonary artery pressures as measured by echocardiography (pulmonary acceleration time). ** reflects significance where p<0.01.

### DEFINITIONS

The term “comprising” as used herein means including principally, but not necessarily solely. Furthermore, variations of the word “comprising”, such as “comprise” and “comprises”, have correspondingly varied meanings.

The terms “subject” and “patient” are used interchangeably herein and include humans and individuals of any species of social, economic or research importance including but not limited to members of the genus ovine, bovine, equine, porcine, feline, canine, primates, rodents.

The terms “treating” and “treatment” as used herein includes administering therapy to prevent, cure, alleviate, ameliorate or prevent the symptoms associated with a disorder, disease, injury or condition.

In the context of this specification the term “polypeptide” means a polymer made up of amino acids linked together by peptide bonds. The polypeptide may be of any length. Except where the context indicates otherwise it will be understood that the term polypeptide also includes peptides and proteins.

The term “at least one” when used in the context of a group of selectable elements includes any one, two or more, up to all members of the group, individually selected and includes any combination of the members of the group. Similarly, the term “at least two” when used in the context of a group of selectable elements includes any selection of two or more members of the group in any combination.

In the context of this specification the terms “redox-induced dysfunction” and “redox-mediated dysfunction” will be understood to have the same meaning.

To the extent that it is permitted, all references cited herein are incorporated by reference in their entirety.

### DETAILED DESCRIPTION AND EXAMPLES

The inventors have surprisingly identified a novel interaction of FXYD1 with endothelial nitric oxide synthase (eNOS) in the endothelium, a critical enzyme in vascular homeostasis, with substantial therapeutic implications for a wide range of acute and chronic vascular disease states.

The FXYD proteins are a family of small type I membrane proteins are named after an invariant FXYD signature sequence in the extracellular domain. The mammalian FXYD (“fixit”) proteins, expressed in a tissue-specific manner (Sweadner and Rael, 2000), are numbered chronologically according to the dates they were cloned. FXYD1 (also referred to as phospholemman) is a small membrane protein expressed in endothelium and vascular smooth muscle. It is known to be functionally associated with the Na+-K+ pump in the heart, where it has been previously demonstrated to have a protective effect against oxidative stress-induced inhibition of the Na+-K+ pump (Bibert et al., 2011). FXYD1 has also been demonstrated to be expressed in vascular smooth muscle cells, and to play a role in acute regulation of the Na+-K+ pump, and contractile tone in response to oxidative stress (Liu et al., 2013).

An example amino acid sequence of human FXYD1 (Palmer et al., 1991) is provided at UniProtKB/Swiss-Prot Accession No. 000168 (SEQ 1) NO. 1)). An example nucleic acid sequence encoding human FXYD1 is EMBL-EBI Accession No. U72245 (SEQ ID No. 2).

eNOS is pivotal in endothelial physiology, regulating vascular tone as well as attenuating platelet aggregation and neutrophil-endothelium interaction (Brunner et al., 2003). However, under conditions of oxidative stress, eNOS becomes “uncoupled”, preferentially producing O₂^{⋅-} and ONOO⁻ which amplifies oxidative stress and exacerbates injury (Burgoyne et al., 2005). Protecting eNOS from uncoupling during pathophysiological insults (e.g.diabetes, hypertension) may halt the amplification process and protect key membrane proteins critical to the health of the artery. The discovery that this uncoupling of the eNOS reductase domain is mediated by glutathionylation (Chen et al., 2010) has led to a paradigm shift in our understanding of eNOS regulation and paves the way for new therapeutic strategies to limit oxidative stress in cardiovascular disease.

As described herein it has now surprisingly been identified by the instant inventors that there is a functional interaction of FXYD1 and eNOS. This is supported by the following experimental results obtained by the instant inventors and described herein.

### Methods

Cell Culture and siRNA Knockdown:

Human coronary artery endothelial cells (HUVECS) were cultured in endothelial growth medium. siRNA against FXYD1 or scrambled control (Qiagen); and FXYD1 expression plasmid were used to examine FXYD1's effect on endothelial cell function and eNOS activity as well as its glutathionylation. Fluorescein-labeled dsRNA oligomer was used to visualize transfection efficiency.

Immunodetection of glutathionylated protein and protein coimmunoprecipitation. To detect glutathionylation of eNOS in co-immunoprecipitation experiments, a commercially available antibody against glutathionylated protein (anti-GSH antibody-Virogen) was used to detect glutathionylation. Aorta were homogenised in ice-cold lysis buffer containing 150 mmol/L NaCl, 50 mmol/L Tris-HCl (pH 8.0), 1% Triton X-100, 2 mM EDTA, and protease inhibitor (Complete EGTA-free, Roche Diagnostics), followed by centrifugation at 16,000 g for 20 min. The supernatant (0.5-1 mg protein) was incubated with the appropriate antibody at a ratio of 1 mg protein and 2.5 μg anti-eNOS antibody (Sigma-Aldrich): 1 mg protein at 4° C. for 1 hour and then with protein A/G-Plus agarose beads. The proteins bound to the collected beads were eluted in Laemmli buffer, subjected to SDS-PAGE and probed with anti-GSH antibody. Western blot chemiluminescence was read by a LAS-4000 image reader and quantified by densitometry using Multi Gauge 3.1 software (Fujifilm Life Science, Tokyo, Japan). Exposure times were adjusted to ensure that the variation in signal intensity was in the linear dynamic range. Standard western blot techniques were used for assessing FXYD1 and eNOS expression.

NO Detection by Confocal Fluorescence Microscopy.

Intracellular NO in HUVECs was detected by using 4-Amino-5-Methylmino-2′,7′-Difluorofluorescein Diacetate (DAF-FM Diacetate) (10 μM, 30 min 37° C.), without pre-incubation with acetylcholine prior to fixation. Nuclei were stained with 4′,6-diamidino-2-phenylindole (DAPI, 1 μM). NO fluorescence was detected using excitation and emission wavelengths of 495 nm and 515 nm respectively and analysed by ImageJ software.

Superoxide Detection by Confocal Fluorescence Microscopy.

Changes in intracellular O₂^{⋅-} were examined in HUVECs loaded with the O₂^{⋅-}-sensitive dye dihydroethidium (DHE) for 30 minutes prior to fixation. Nuclei were stained with 4′,6-diamidino-2-phenylindole (DAPI, 1 μM) and cells mounted on glass slides with Vectashield mounting medium and viewed with a confocal microscope (Leica TCS SP5).

Echocardiography in FXYD1 KO and WT mice: Transthoracic echocardiography was obtained in lightly sedated mice (isofluranel %) breathing spontaneously using a 30-MHz transducer (Vevo 770; Visualsonics, Toronto, Ontario, Canada). We measured Pulmonary Artery Acceleration Time (PAT) to noninvasively estimate pulmonary arterial pressure before (baseline) and after exposure of mice to a hypoxic environment for 3 and 6 weeks.

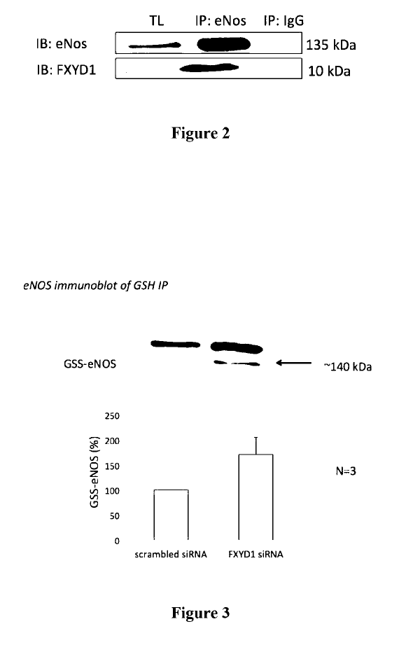

FXYD1 is expressed and physically interacts with eNOS as demonstrated by coimmunoprecipitation assays in human endothelial cells (HUVECs; FIG. 2).

Silencing of endogenous FXYD1 expression in human endothelial cells leads to increased glutathionylation of eNOS (FIG. 3). As this is the molecular mechanism of redox uncoupling of eNOS, this is predicted to reduce NO bioavailability, and increase superoxide production.

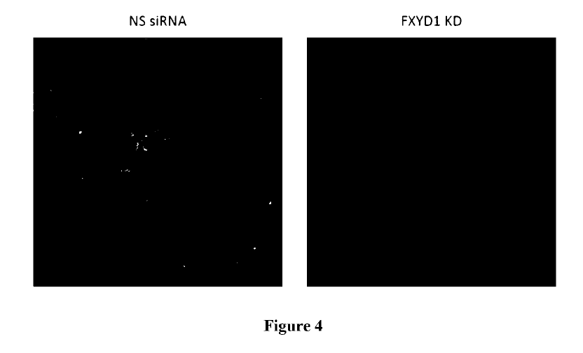

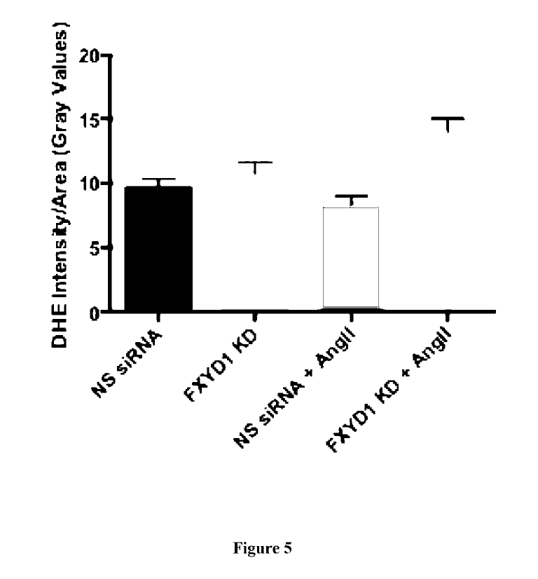

Silencing of endogenous FXYD1 expression in human endothelial cells leads to substantial reduction in NO bioavailability at baseline; when stimulated by acetylcholine (FIG. 4 and FIG. 5); and under conditions of higher oxidative stress driven by exposure to the neurohormone Angiotensin II.

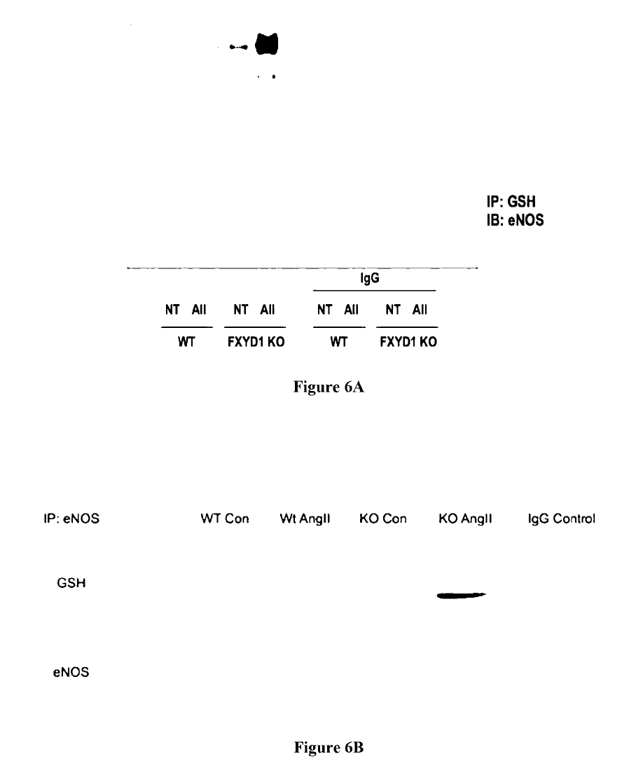

Knockout of FXYD1 (in vivo-knockout mouse) results in increased glutathionylation of eNOS under baseline conditions, and augments the susceptibility of eNOS to Angiotensin II induced glutathionylation in the heart (FIG. 6A) and the aorta (FIG. 6B). This further supports the protective role of FXYD1 against redox damage to critical membrane proteins in the cardiovascular system.

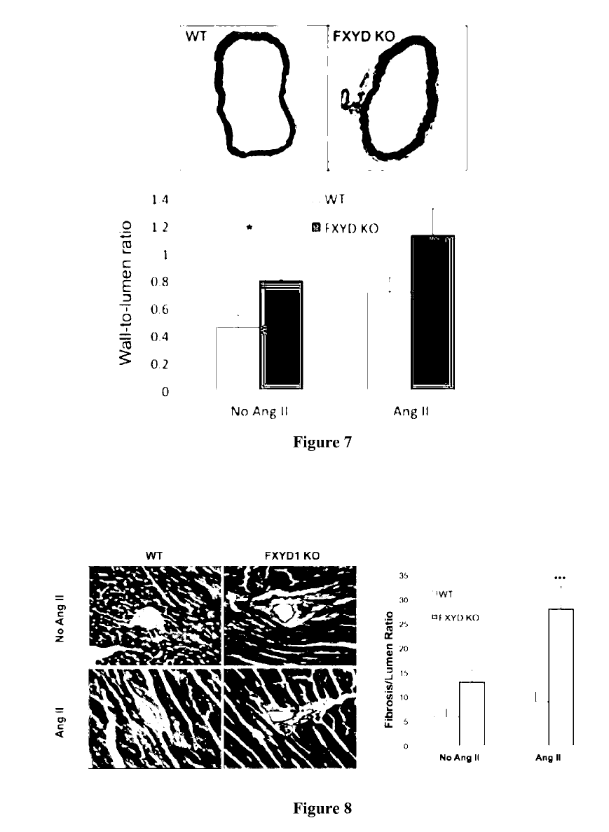

Knockout of FXYD1 (in vivo-knockout mouse) results in chronic vascular changes of medial hypertrophy and perivascular fibrosis, consistent with eNOS uncoupling, and excess ROS production. These phenotypes are exacerbated by Angiotensin II administration (FIG. 7 and FIG. 8).

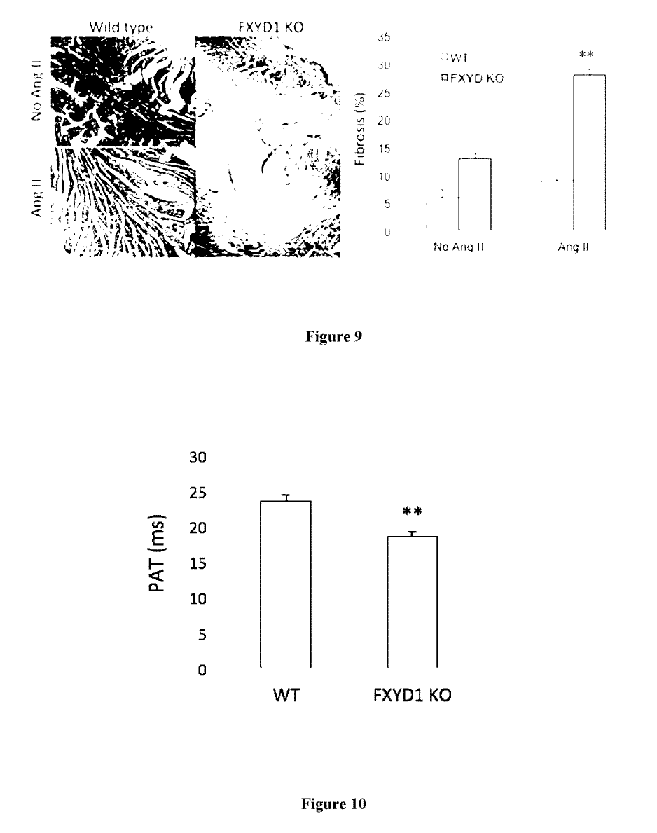

Knockout of FXYD1 (in vivo knockout mouse) results in myocardial interstitial fibrosis, and exacerbation of Angiotensin II-induced myocardial fibrosis (FIG. 9). The inventors consider that the mechanism of this may relate to altered myocardial ROS production secondary to improved eNOS coupling.

The endogenous protective role of FXYD1 against eNOS uncoupling pointed to a potential role in regulation of pulmonary arterial vasomotor tone as well as vascular remodelling in the pulmonary vasculature. In vivo studies in knockout mice demonstrate an elevated pulmonary arterial pressure as reflected by echocardiographic measurements of pulmonary acceleration time (PAT, as shown in FIG. 10, n=7 per mice).

Thus, this new data indicates that the role of FXYD1 in vascular cells is not limited to maintenance of Na⁺ pump function, but also includes the protection of eNOS against oxidative stress-induced glutathionylation and uncoupling. This has important acute and chronic implications to vascular health. The identification of this functional interaction has therapeutic implications for a wide range of vascular disease states. The finding that FXYD1 silencing also results in increased myocardial fibrosis points to a possible beneficial effect of targeted therapies in myocardial disease states characterized by ROS-dependent myocardial fibrosis.

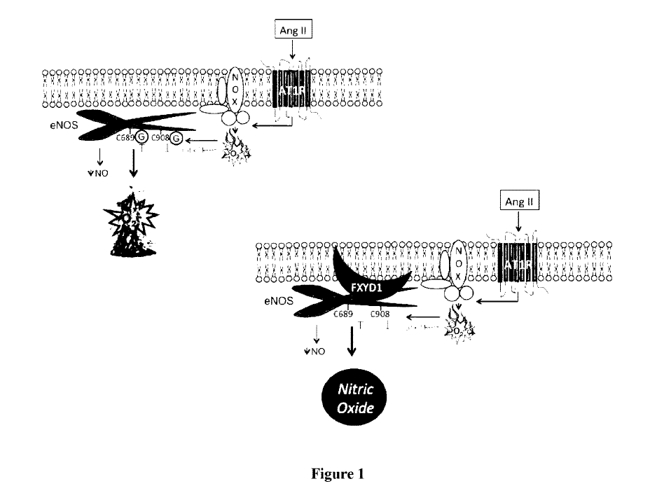

Based on the findings disclosed herein, delivery of FXYD1 to the endothelium is predicted to provide protection against redox induced uncoupling, and thus halt or ameliorate the “bonfire” positive feedback of reactive oxygen species-induced production of reactive oxygen species (as shown in FIG. 1). This is a key, integrating step in the pathophysiology of vascular disease.

Three delivery approaches are apparent with variable suitability to different clinical scenarios, as follows.

Delivery of Recombinant FXYD1 or Engineered Derivatives.

The feasibility of achieving spontaneous membrane insertion of the recombinant lipophilic protein is supported by published findings that: (1) rFXYD proteins spontaneously inserts in the correct orientation in artificial membranes (assessed by Nuclear Magnetic Resonance; Teriete et al., 2007); FXYD proteins associate with Na⁺ pump subunits (Crambert et al., 2004); (2) exogenous, purified FXYD1 partitions into membrane fragments and associates with the Na⁺ pump within 5 min as indicated by its co-immunoprecipitation and cysteine cross-linking with a subunit (Mahmmoud et al., 2000; Cornelius et al., 2005); and (3) incubation of cardiac myocytes in solution containing rFXYD3 proteins (used for purposes of immunodetection as not endogenous to heart) results in functional association of rFXYD3 protein with the Na⁺ pump (Bibert et al., 2011).

Administration of recombinant FXYD1 protein is most appropriate for situations where acute reversal of endothelial oxidative stress is beneficial, and, ideally, where the vessel is directly accessible for delivery such as in surgical or interventional procedures.

Examples include incubating coronary artery bypass grafts prior to anastomosis during surgery; coating of vascular stents, including coronary artery stents; delivery to peripheral vascular bypass grafts particularly in the setting of ischaemic diabetic feet; delivery to arterial-venous fistulas in chronic renal failure patients either at time of initial surgery, or during surgical correction of occluded fistulas; and intra-arterial delivery to protect either the coronary or brain circulation from ischaemia-reperfusion injury in the setting of acute myocardial infarction or stroke.

Gene Transfer.

Delivery of viral vector of FXYD1 is feasible for the above applications.

Transcriptional Regulation.

Identifying factors involved in FXYD1 expression would allow therapy to be developed more suited to chronic vascular disease, such as systemic arterial disease in diabetic or hypertensive patients, or those who smoke.

In each case of therapeutic use of the FXYD1 or derivative thereof a therapeutically effective amount of the FXYD1 or the derivative thereof will typically be administered to the subject. The term “therapeutically effective amount” as used herein includes within its meaning a sufficient amount of a compound or composition to provide a desired therapeutic effect. The exact amount required will vary from subject to subject depending on factors such as the species being treated, the age and general condition of the subject, co-morbidities, the severity of the condition being treated, the particular agent being administered and the mode of administration. Thus, for any given case, an appropriate “therapeutically effective amount” may be determined by one of ordinary skill in the art using only routine methods. Similarly, where the therapy comprises the administration of a precursor of the active agent, such as the administration of a genetic construct that is intended to express the therapeutic agent, for example the FXYD1 or derivative thereof, it will be understood that the genetic construct so-delivered will be capable of providing a therapeutically effective amount of the active.

Treatment of a subject with FXYD1 or a derivative thereof capable of interacting with eNOS according to the invention may be the sole treatment given to the subject or may be one component of a combined regime treatment for the subject in which the FXYD1-based treatment is combined with other treatments for the condition. The term “combined with” and similar terms such as “in conjunction with” as used herein in relation to a therapeutic regime means that each of the drugs and other therapeutic agent(s) is used in the treatment of a subject and that each of the drugs and other therapeutic agents in the “combined” therapeutic regime may be administered to the subject simultaneously with one or more of the other agents in the therapeutic regime, or may be administered to the subject at a different time to one or more of the other agents in the therapeutic regime.

It will be understood that the term “combined with” and similar terms such as “in conjunction with” as used herein in relation to a therapeutic regime encompasses within their meaning administration of each of the drug(s) and other therapeutic agent(s) via different modes (for example one may be administered orally and another by injection). The term “combined with” and similar terms such as “in conjunction with” when used in relation to a therapeutic regime may mean that any one or more of the drugs or other agents may be physically combined prior to administration to the subject, and it will be understood that the term also includes administration of the one or more drugs and other therapeutic agents as separate agents not in prior physical combination.

On the basis of the disclosure herein that FXYD1 protects the endothelium from eNOS uncoupling under conditions of oxidative stress, the invention provides novel therapeutic strategies for patients with arterial disease, for example by way of delivery of FXYD1, or engineered or naturally occurring derivatives or variants thereof, to such patients.

The term “derivative” when used in relation to an FXYD1 protein of the present invention includes any functionally equivalent FXYD1 protein including any fusion molecules produced integrally (e.g., by recombinant means) or added post-synthesis (e.g., by chemical means). Such fusions may comprise FXYD1 proteins of the invention conjugated to a polypeptide (e.g., puromycin or other polypeptide), a small molecule (e.g., psoralen) or an antibody. As described herein the inventors have demonstrated that FXYD1 interacts with eNOS and in so doing plays a role in the protection of eNOS against oxidative stress-induced glutathionylation and uncoupling.

In this context it will be understood that a fusion protein or polypeptide may comprise a plurality of FXYD proteins of the invention, such as a polypeptide where two or more FXYD proteins are present on a single polypeptide.

A fusion protein or polypeptide comprising one or more FXYD1 proteins of the invention may additionally comprise one or more unrelated sequences. Such a sequence will generally be referred to herein, in the context of a fusion protein or polypeptide, as a “fusion partner”. Typically, a fusion partner is an amino acid sequence, and may be a polypeptide. A fusion partner may, for example, be selected to assist with the production of the peptide or peptides. Examples of such fusion partners include those capable of enhancing recombinant expression of the peptide or of a polypeptide comprising the peptide; those capable of facilitating or assisting purification of the peptide or a polypeptide comprising the peptide such as an affinity tag. Alternatively, or in addition, a fusion partner may be selected to increase solubility of the peptide or of a polypeptide comprising the peptide, to increase the immunogenicity of the peptide, to enable the peptide or polypeptide comprising the peptide to be targetted to a specific or desired intracellular compartment.

Methods for the preparation of fusion proteins are known in the art. Typically, a fusion protein may be made by standard techniques such as chemical conjugation, peptide synthesis or recombinant means. A fusion protein may include one or more linker(s), such as peptide linker(s), between component parts of the protein, such as between one or more component peptides, and/or between one or more fusion partners and/or component peptides. Such a peptide linker (s) may be chosen to permit the component parts of the fusion protein to maintain or attain appropriate secondary and tertiary structure.

FXYD proteins of the invention may be prepared by any suitable means, such as by isolation from a naturally occurring form, by chemical synthesis or by recombinant means. The skilled addressee will be aware of standard methods for such preparation, such as by isolation from a naturally occurring longer amino acid sequence by enzymatic cleavage, such as by chemical synthesis, such as by recombinant DNA technology.

The FXYD protein of the invention, or a fusion protein or polypeptide comprising an FXYD protein of the invention as a component part thereof may be a soluble peptide, fusion protein or polypeptide.

The invention also encompasses the use of a “variant” of FXYD L The term “variant” when used in relation to an FXYD1 protein of the present invention includes any functionally equivalent FXYD1 protein. The term “variant” also encompasses a functional fragment of an FXYD1 protein or polypeptide or oligopeptide and also encompasses functional homologues. For example, whilst the invention is described herein with reference to a specific FXYD1 amino acid sequence or polynucleotide sequence (such as represented in SEQ ID NOs: 1 and 2), it will be understood that there may exist or be synthesised FXYD1 polypeptides or polynucleotides that have, or encode, a capability of interacting with eNOS in a manner similar to that of FXYD1 to effect a degree of protection of eNOS against oxidative stress-induced glutathionylation and uncoupling.

Variants or homologues may have one or more amino acid substitutions, deletions, additions and/or insertions in the amino acid sequence. Variants or homologues of FXYD1 proteins of the invention preferably exhibit at least about 70%, at least about 80%, at least about 85%, or at least about 90% identity to a native FXYD1 protein, more preferably at least about 92%, or at least about 94% identity, or at least about 95% identity, or at least about 96% identity, or at least about 97% identity, or at least about 98% identity, or at least about 99% identity across the length of the variant or homologue to a native FXYD1 protein.

FXYD1 protein or variants may be modified by, for example, the deletion or addition of amino acids. Amino acid substitutions include, but are not necessarily limited to, amino acid substitutions known in the art as “conservative”. A “conservative” substitution is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes.

Typically, a variant FXYD1 protein differs from a native FXYD1 protein by substitution, deletion or addition of five amino acids or fewer, such as by four, or three, or two, or one amino acids.

Typically, an FXYD1 protein of the invention is an isolated protein. It will be understood that the term “isolated” in this context means that the protein has been removed from or is not associated with some or all other components with which it would be found in the natural system. For example, an “isolated” peptide may be removed from other amino acid sequences within an FXYD1 polypeptide sequence, or may be removed from natural components such as unrelated proteins. For the sake of clarity, an “isolated” FXYD1 protein includes an FXYD1 protein which has been chemically synthesised and includes a polypeptide or oligopeptide which has been prepared by recombinant methods. As described herein the isolated FXYD1 protein of the invention may be included as a component part of a longer polypeptide or fusion protein.

Accordingly, a “derivative” or “variant” of FXYD1 as used herein incorporates a functional requirement that it be capable of interacting with eNOS, thereby playing a role in the protection of eNOS against oxidative stress-induced glutathionylation and uncoupling. It will be understood that “functionally equivalent” in the context of “derivatives” and “variants” does not require the identical quantitative result as may be seen with FXYD1. A derivative or variant may be more active or may be less active in achieving a desired outcome compared to a reference FXYD1.

It will be understood that throughout this specification reference to the use of FXYD1 in a therapeutic method, or in the context of such use (for example reference to a pharmaceutical composition of FXYD1 for use in therapy), is reference not only to FXYD1 but also to derivatives, variants, fragments, etc thereof. It will simply be the case that for brevity of language rather than of meaning, the term FXYD1 may be used.

The invention provides polynucleotides that encode one or more FXYD1 protein(s) of the invention and polynucleotides that encode one or more fusion protein(s) or polypeptide(s) comprising FXYD1 protein(s) of the invention, as described herein. In certain embodiments of the invention, polynucleotide sequences or fragments thereof which encode peptides of the invention, or fusion proteins or functional equivalents thereof, may be used in recombinant DNA molecules to direct expression of a polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be recognized by the skilled artisan, polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials. Polynucleotides may comprise a native sequence (i.e., an endogenous sequence that encodes an FXYD1 protein or a portion thereof) or may comprise a variant, or a biological or antigenic functional equivalent of such a sequence. Polynucleotide variants may contain one or more substitutions, additions, deletions and/or insertions, as further described below, preferably such that the immunogenicity of the encoded polypeptide is not diminished, relative to a native FXYD1 protein. The effect on the immunogenicity of the encoded polypeptide may generally be assessed as described herein. The term “variants” also encompasses homologous genes of xenogenic origin.

In order to express a desired polypeptide, the nucleotide sequences encoding the peptide, fusion protein or polypeptide, or functional equivalents, may be inserted into an appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y. and updated editions thereof.

The invention thus provides vectors comprising a polynucleotide sequence of the invention. In one embodiment the vector may be an expression vector. The invention also provides a host cell comprising a polynucleotide or vector of the invention. The invention also provides methods for the preparation of a peptide of the invention, such a method comprising culturing a host cell comprising a polynucleotide or expression vector of the invention under conditions conducive to expression of the encoded peptide. In one embodiment, the method further comprises purifying the expressed peptide.

In the performance of the methods of the invention the FXYD1 is typically brought into contact with eNOS or caused to be brought into contact with eNOS to effect a degree of protection of eNOS against oxidative stress-induced glutathionylation and uncoupling. Alternatively or in addition, the FXYD1 which is endogenous to the cell or tissue may be altered by the administration of an agent or agents capable of increasing or decreasing the expression for the endogenous FXYD1, such that, for example, additional FXYD1 is caused to be in contact with eNOS. “Contact” or “contacting” as used herein refers to exposing tissue, organs or cell to an FXYD1 protein(s) or prodrugs of the invention so that it can interact with eNOS. Contacting may be in vitro, for example by adding the FXYD1 protein or prodrug to cultured tissue or cells or vascular tissue or graft for diagnostic or research purposes or to test for susceptibility of the tissue or cells or vascular tissue or graft to the FXYD1 protein or prodrug. Contacting may be in vivo, for example administering the FXYD1 protein or prodrug to a subject, such as for treatment or prevention of an undesirable condition. Contacting may be ex vivo such as by exposing a vascular graft to a composition comprising FXYD1.

As described herein administering FXYD1 to a subject may be in any appropriate form or manner, such as by administering a pharmaceutical composition comprising FXYD1, administering a vascular graft which has been exposed to FXYD1, such as by soaking the graft in a composition comprising FXYD1, administering a precursor of FXYD1 protein, such as a prodrug or a nucleic acid sequence encoding FXYD1 in a vector capable of expressing FXYD1, administering a medical device such as a stent capable of delivering FXYD1.

Devices, such as stents, for delivery of therapeutic substances to vasculature are known in the art. As a result a medical device such as a stent capable of delivering FXYD1 may be prepared by any appropriate method. Suitable methods may be found for example in U.S. Pat. No. 5,697,967 entitled “Drug-eluting stent”; T. Cooper Woods and Andrew R. Marks, Drug-Eluting Stents, Annual Review of Medicine, Vol. 55: 169-178; U.S. Pat. No. 7,135,038 entitled “Drug eluting stent”; the contents of each of which are incorporated herein by reference.

FXYD proteins are lipid- and hence membrane-soluble. Thus administration of FXYD1 proteins in the setting of acute ischaemic injury may be used, e.g. FXYD1 in the treatment of for example infarction and ischaemia reperfusion injury. The proteins may be administered intravenously, or directly infused into an infarct-related artery for example in the setting of acute intervention with angioplasty procedures. In other embodiments an infusion of an FXYD1 protein may be administered on a time scale of minutes to hours.

In some embodiments FXYD1 proteins of the present invention may be produced with or conjugated to proteins, polypeptides, oligopeptides, antibodies or fragments thereof, radioactive particles, nanoparticles or microparticles.

As described herein the methods of the invention include the use of an agent or agents capable of increasing the levels of functional FXYD1 in a cell, thereby influencing the amount of functional FXYD1 that may be caused to interact with eNOS. Methods of the invention also encompass the use of small molecules to improve functional interaction of FXYD1 and eNOS. Methods are known in the art for the use of, for example, small molecules that influence the expression of a target gene, such that the expression of the target gene is increased or decreased as the desired case may be. Typically, in the methods of the invention the target gene encodes FXYD1 or an endogenous regulator of FXYD1 expression. Typically, the invention comprises increasing the expression of FXYD1 in a target cell or tissue. Regulation of the amount of a target gene product can also be influenced at the level of post-transcriptional processing or translation. Methods of the invention also encompass the use of microRNA that might influence regulation of FXYD1 expression. miRNAs are also known to be involved in heart and cardiovascular disease and the methods of the invention therefore also envisage the use of microRNA, siRNA, antisense, ribozyme, or shRNA constructs.

The methods of the invention may be in vivo, ex vivo or in vitro methods. An example of an in vitro method is a method for research or development purposes. An example of an in vivo method is a method of treating or preventing a disease in a patient requiring said treatment or prevention.

In one embodiment, the method comprises treatment of diabetic peripheral vascular disease (where treatment may be directly administered, by both soaking surgically treated vessel or grafts, and by injecting intra-arterially to relevant zone). In one embodiment the method comprises treatment of a patient undergoing coronary artery bypass surgery (where grafts can be directly incubated). In one embodiment the method comprises treatment of a patient receiving arterial stents, for example for coronary, peripheral or brain circulation, where stents can be coated with FXYD1 delivery agent.

In one embodiment the method comprises treatment of a chronic renal failure patient with arterial-venous (also known as arteriovenous) fistulas required for dialysis, where failure of the fistula is a major clinical problem. FXYD1 delivery is predicted to be beneficial if delivered at time of fistula creation, or at time of surgical intervention for occlusion.

In one embodiment the method comprises treatment of a patient suffering from acute myocardial infarction, reducing myocardial ischaemia-reperfusion injury which is driven, in part, by endothelial dysfunction.

In one embodiment the method comprises treatment of a patient suffering from acute cerebrovascular accident (stroke), in whom ischaemia-reperfusion injury is driven by endothelial dysfunction.

In one embodiment the method comprises treatment of a patient with chronic vascular disease, where treatment to increase expression of FXYD1 in the vasculature is predicted to reduce vascular events.

In one embodiment the method comprises treatment of a patient with pulmonary hypertension (such as idiopathic, or secondary to scleroderma or related connective tissue disease states), characterized by eNOS uncoupling, perivascular fibrosis, and hypertrophy of the media. In this case, inhalation of gene therapy is the delivery method of choice, successful in other pulmonary disease states.

The most appropriate treatment regime for any particular patient may be determined by the treating physician and will depend upon a variety of factors including: the disorder being treated and the severity of the disorder; activity of the compound or agent employed; the composition employed; the age, body weight, general health, sex and diet of the patient; the time of administration; the route of administration; the rate of sequestration of the agent or compound; the duration of the treatment; drugs used in combination or coincidental with the treatment, together with other related factors well known in medicine.

In one aspect of the present invention, the administration of FXYD1 may be as an “add-on”, in which a patient may be treated with a conventional drug. For example a FXYD1 may be administered before, during or after a treatment with, for example a more conventional theraputic drug or regimen for vascular disease. Consequently, it will be appreciated that in this context the term “add-on” refers to an additional therapeutic integer (the FXYD1); it does not mean that the FXYD1 must be added as the last drug. The order and composition of the specific drugs and drug classes in the combination therapy may be determined by the skilled addressee, and may include, for example where the therapeutic regime involves the administration of multiple drug classes, the FXYD1 may be administered at any stage during the regimen.

As a further example of a treatment regime of the method of the invention, the condition of a patient suffering a myocardial infarction may be at least partially stabilized prior to administration of the FXYD1. Furthermore, the condition of a patient may be at least partially stabilized prior to commencement of a method of the invention. Either of such treatment regimes may be referred to as first stabilizing a patient.

As a further example of a treatment regime, FXYD1 may be the first drug to be administered in the treatment of the vascular disease without any prior medication or stabilisation. The FXYD1 proteins proposed for the present invention may be administered as compositions either therapeutically or preventively. In a therapeutic application, compositions are administered to a patient already having a condition characterised by redox-induced dysfunction of the vasculature, in an amount sufficient to effectively treat the patient.

The therapeutically effective dose level for any particular patient will depend upon a variety of factors including: the disorder being treated and the severity of the disorder; activity of the compound or agent employed; the composition employed; the age, body weight, general health, sex and diet of the patient; the time of administration; the route of administration; the rate of sequestration of the agent or compound; the duration of the treatment; drugs used in combination or coincidental with the treatment, together with other related factors well known in medicine. One skilled in the art would be able, by routine experimentation, to determine an effective, amount of the FYXD1 protein, and other agents where appropriate, which would be required to treat the condition.

Further, it will be apparent to one of ordinary skill in the art that the optimal quantity and spacing of individual dosages and, where combination therapy is used, optimal quantity and spacing of administration of the various agents of the combination therapy, will be determined by the nature and extent of the disease condition or state being treated, the form, route and site of administration, and the nature of the particular individual being treated. Also, such optimum conditions can be determined by conventional techniques.

In general, suitable compositions comprising FXYD1 may be prepared according to methods which are known to those of ordinary skill in the art and accordingly may include a pharmaceutically acceptable carrier, diluent, excipient and/or adjuvant.

These compositions can be administered by standard routes. In general, the compositions may be administered by the parenteral (e.g., intravenous, intraspinal, subcutaneous or intramuscular) route. The compositions may be administered by injecting intra-arterially to relevant zone. The compositions may be administered by, for example, exposing vasculature to a composition comprising FXYD1, such as by soaking a vascular graft in a composition comprising FXYD1 The FXYD1 may be administered by use of a medical device, such as a stent comprising FXYD1, which may be a coated stent, wherein the coating comprises FXYD1, in which case the composition may be used for the preparation of the medical device. The compositions comprising FXYD1 may therefore be in a form suitable for, for example, coating onto or into a stent, or for contacting vasculature, such as by soaking or infusing into a vascular graft prior to implantation.

The carriers, diluents, excipients and adjuvants must be “acceptable” in terms of being compatible with the other ingredients of the composition, and not deleterious to the recipient thereof. Examples of pharmaceutically acceptable carriers or diluents are demineralised or distilled water; saline solution; vegetable based oils such as peanut oil, safflower oil, olive oil, cottonseed oil, maize oil, sesame oils, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; DMSO, N, N-dimethylacetamide (DMA), lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

The composition may include agents which increase the bioavailability or therapeutic duration of the active compound or compounds.

The compositions of the invention may be in a form suitable for parenteral administration, such as, subcutaneous, intramuscular or intravenous injection or infusion.

For administration as an injectable solution or suspension, non-toxic parenterally acceptable diluents or carriers can include, Ringer's solution, isotonic saline, phosphate buffered saline, ethanol and 1,2 propylene glycol.

Adjuvants typically include emollients, emulsifiers, thickening agents, preservatives, bactericides and buffering agents.

As FXYD1 proteins are lipid soluble the compositions may also be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which is incorporated herein by reference.

The invention has been described herein with reference to experimental results. The experiments are intended to serve to illustrate this invention and should not be construed as limiting the generality of the disclosure of the description throughout this specification.

To the extent that it is permitted, all references cited herein are incorporated by reference in their entirety.

### REFERENCES

Bibert S, Liu C C, Figtree G A, Garcia A, Hamilton E J, Marassi F M, Sweadner K J, Cornelius F, Geering K, Rasmussen H H. Fxyd proteins reverse inhibition of the na⁺-k⁺ pump mediated by glutathionylation of its β₁ subunit. J Biol Chem. 2011; 286:18562-18572.
Liu C-C, Karimi Galougahi K, Weisbrod R M, Hansen T, Ravaie R, Nunez A, Liu Y B, Fry N, Garcia A, Hamilton E J, Sweadner K J, Cohen R A, Figtree G A. Oxidative inhibition of the vascular na+-k+ pump via nadph oxidase-dependent β1-subunit glutathionylation: Implications for angiotensin ii-induced vascular dysfunction. Free Radical Biology and Medicine. 2013; 65:563-572.
Brunner F, Maier R, Andrew P, Wolkart G, Zechner R, Mayer B. Attenuation of myocardial ischemia/reperfusion injury in mice with myocyte-specific overexpression of endothelial nitric oxide synthase. Cardiovascular Research. 2003; 57:55-62.
Burgoyne J R, Mongue-Din H, Eaton P, Shah A M. Redox signaling in cardiac physiology and pathology. Circ Res. 2012; 111:1091-1106.
Chen C A, Wang T Y, Varadharaj S, Reyes L A, Hemann C, Talukder M A, Chen Y R, Druhan L J, Zweier J L. S-glutathionylation uncouples enos and regulates its cellular and vascular function. Nature. 2010; 468:1115-1118.
Teriete P, Franzin C M, Choi J, Marassi F M. Structure of the na,k-atpase regulatory protein fxydl in micelles. Biochemistry. 2007; 46:6774-6783.
Crambert G, Li C, Swee L K, Geering K. Fxyd7, mapping of functional sites involved in endoplasmic reticulum export, association with and regulation of na,k-atpase. J Biol Chem. 2004; 279:30888-30895.
Mahmmoud Y A, Vorum H, Cornelius F. Identification of a phospholemman-like protein from shark rectal glands. Evidence for indirect regulation of na,k-atpase by protein kinase c via a novel member of the fxyd family. J Biol Chem. 2000; 275:35969-35977.
Cornelius F, Mahmmoud Y A, Meischke L, Cramb G. Functional significance of the shark na,k-atpase n-terminal domain. Is the structurally variable n-terminus involved in tissue-specific regulation by fxyd proteins? Biochemistry. 2005; 44:13051-13062.
Galougahi K K, Liu C C, Gentile C, Kok C, Nunez A, Garcia A, Fry N A, Davies M J, Hawkins C L, Rasmussen H H, Figtree G A. Glutathionylation mediates angiotensin ii-induced enos uncoupling, amplifying nadph oxidase-dependent endothelial dysfunction. Journal of the American Heart Association. 2014; 3:e00073 1.
Palmer, C. J., Scott, B. T., and Jones, L. R. (1991). Purification and complete sequence determination of the major plasma membrane substrate for cAMP-dependent protein kinase and protein kinase C in myocardium. J Biol Chem 266, 11126-11130.
Sweadner, K. J., and Rael, E (2000). The FXYD gene family of small ion transport regulators or channels: cDNA sequence, protein signature sequence, and expression. Genomics 68, 41-56.

1. A method for the treatment or prevention of redox-induced dysfunction of the vasculature, the method comprising administering to a patient having or at risk of redox-mediated dysfunction of the vasculature a therapeutically effective amount of FXYD1 or a derivative or variant thereof capable of interaction with endothelial nitric oxide synthase.

2. The method according to claim 1, wherein the patient has a condition associated with endothelial dysfunction.

3. The method according to claim 1, wherein administering FXYD1 or a derivative or variant thereof capable of interaction with endothelial nitric oxide synthase comprises (i) delivery of a nucleic acid sequence encoding FXYD1 or a derivative or variant thereof capable of interaction with endothelial nitric oxide synthase to selected vasculature of said patient; or (ii) delivery of a viral vector to selected vasculature of said patient, the viral vector encoding FXYD1 or a derivative or variant thereof capable of interaction with endothelial nitric oxide synthase.

4. (canceled)

5. The method according to claim 1, wherein the method comprises administration of said FXYD1 or a derivative or variant thereof capable of interaction with endothelial nitric oxide synthase to a vessel during surgical or interventional procedure on said patient.

6. The method according to claim 1, wherein administration to said patient comprises incubating, in a composition comprising FXYD1 or a derivative or variant thereof capable of interaction with endothelial nitric oxide synthase, a coronary artery bypass graft prior to anastomosis during surgery.

7. The method according to claim 1, wherein administration to said patient comprises implantation in said patient of a coated vessel or coated stent, said coating comprising FXYD1 or a derivative or variant thereof capable of interaction with endothelial nitric oxide synthase (eNOS).

8. The method according to claim 1, wherein the patient has a condition selected from the group consisting of myocardial infarction, diabetes, such as diabetic peripheral vascular disease, coronary artery disease, dysfunction in the coronary, peripheral or brain circulation, chronic renal failure, such as with arterial-venous fistulas, acute cerebrovascular accident (stroke), ischaemia-reperfusion injury, chronic vascular disease, pulmonary hypertension, neuromuscular disease.

9. The method according to claim 1, wherein said patient is undergoing coronary artery bypass surgery.

10. The method according to claim 1, wherein treatment comprises placement of one or more arterial stent(s).

11. The method according to claim 1, wherein said vasculature is coronary, peripheral or brain circulation vasculature.

12. The method according to claim 1, wherein said treatment is of arterial-venous fistulas required for dialysis in a patient having chronic renal failure.

13. The method according to claim 1, wherein said patient has pulmonary hypertension (such as idiopathic, or secondary to scleroderma or related connective tissue disease states), characterized by eNOS uncoupling, perivascular fibrosis, and hypertrophy of the media.

14. A medical device for implantation in a patient undergoing vasculature therapy, the device comprising (i) a therapeutic amount of FXYD1 or a derivative or variant thereof capable of interaction with endothelial nitric oxide synthase; or (ii) a nucleic acid sequence encoding FXYD1 or a derivative or variant thereof capable of interaction with endothelial nitric oxide synthase.

15. (canceled)

16. The medical device according to claim 13, wherein said device comprises a naturally-occurring component(s).

17. The medical device according to claim 13, wherein said device comprises vascular tissue or blood vessel.

18. The medical device according to claim 13, wherein said device comprises a synthetic component(s).

19. The medical device according to claim 13, wherein said device is, or comprises, a coated vascular stent, such as a coronary artery stent.

20. The medical device according to claim 13, wherein said device comprises a manufactured stent, a vascular graft, or a combination thereof.

21. The medical device according to claim 13, wherein said device comprises a drug-eluting stent or graft.

22. FXYD1 or a derivative thereof capable of interaction with endothelial nitric oxide synthase, or a composition comprising said FXYD1 or said derivative thereof, for the treatment or prevention of redox-induced dysfunction of the vasculature.

23. (canceled)

24. (canceled)

25. (canceled)
